(19) United States
(12) Patent Application Publication
(10) Pub. No.: US 2022/0132902 A1
(43) Pub. Date: May 5, 2022

(54) PREBIOTIC COMPOSITION OF PECTIN, BETA-GLUCAN, XYLOOLIGOSACCHARIDE AND/OR ASHWAGANDHA AND A METHOD OF IMPROVING MOOD

(71) Applicant: Pharmavite, LLC, West Hills, CA (US)

(72) Inventors: Vandana Sharma, San Diego, CA (US); Susan Hazels Mitmesser, Agoura Hills, CA (US)

(73) Assignee: Pharmavite, LLC, West Hills, CA (US)

(21) Appl. No.: 17/400,077

(22) Filed: Aug. 11, 2021

Related U.S. Application Data
(60) Provisional application No. 63/064,733, filed on Aug. 12, 2020.

(51) Int. Cl. A23L 33/125; A61K 36/81; A61K 31/717; A61K 31/732; A23L 33/105

(52) U.S. Cl. CPC A23L 33/125; A61K 36/81; A23L 33/105; A61K 31/732; A61K 31/717

(57) ABSTRACT

A composition and a method for improving mood, particularly reducing stress, anxiety and/or depression including administering a composition including at least two prebiotics selected from a source of pectin, a source of beta-glucan, a source of xylooligosaccharide and Ashwagandha. The composition may be in the form of a supplement that is added to a diet (supplements a normal human diet). The composition includes the at least two prebiotics in an amount to increase production of (A) tryptophan or (B) tryptophan and/or at least one of (i) indole, (ii) an indole derivative, (iii) 2,3-pyridinecarboxylic acid (iv) 3-(4-hydroxyphenyl) propionic acid and (v) a short chain fatty acid or (C) any other metabolite (e.g., dopamine, serotonin, GABA) relevant to (eg. benefitting) brain health such as improving mood.

### CROSS-REFERENCE TO RELATED APPLICATION

This disclosure claims the benefit of the filing date of United States Provisional Patent Application No. 63/064,733, filed on Aug. 12, 2020, the entire contents of all of which are hereby expressly incorporated by reference.

### FIELD

Dietary supplement composition.

### BACKGROUND

A prebiotic is a substrate that is selectively utilized by host microorganisms to confer a health benefit to the body. An example is a function of a prebiotic to induce the growth or activity of beneficial microorganisms such as bacteria. Prebiotics have been postulated to beneficially alter the composition of organisms in the gut microbiome by acting as a substrate for the growth or activity of advantageous bacteria that colonize the large bowel. Prebiotics and fibers may be indigestible and reach the colon intact where they are metabolized by resident colonic microbes (also known as gut microbiota), which stimulates growth of select beneficial bacteria and production of health promoting metabolites.

### BRIEF DESCRIPTION OF THE DRAWINGS

FIG. 1 shows the effect on gut microbial tryptophan production when human gut microbiota was individually cultured with oats beta-glucan, XOS and apple pectin;

FIG. 2 shows the effect on gut microbial indole production when human gut microbiota was cultured with apple pectin;

FIG. 3 shows the effect on gut microbial production of two indole derivatives and two other microbial metabolites when human gut microbiota was cultured with beta-glucan; and

FIG. 4 shows the effect on gut microbial short chain fatty acid production when human gut microbiota was cultured individually with apple pectin, beta-glucan, xylooligosaccharides and Ashwagandha;

FIG. 5 shows corticosterone levels in female mice after the mice underwent a forced swim test (FST) as described in Example 2;

FIG. 6a shows serum cytokines mILa, mILb, mIL-2 and mIL-3 levels in male and female mice on unsupplemented diets or diets supplemented with one or more prebiotic or maltodextrin after the mice underwent acute stress as described in Example 2;

FIG. 6b shows serum cytokines mIL-4, mIL-5, mIL-6 and mIL-10 levels in male and female mice on unsupplemented diets or diets supplemented with one or more prebiotic or maltodextrin after the mice underwent acute stress as described in Example 2;

FIG. 6c shows serum cytokines (mIL-12, mIL-17, mMCP-1 and mIFNγ levels in male and female mice on unsupplemented diets or diets supplemented with one or more prebiotic or maltodextrin after the mice underwent acute stress as described in Example 2;

FIG. 6d shows serum cytokines mTNFa, mMIP-1a, mGM-CSF and mRANTES levels in male and female mice on unsupplemented diets or diets supplemented with one or more prebiotic or maltodextrin after the mice underwent acute stress as described in Example 2;

FIG. 7 shows anxiety like behavior in the elevated plus maze (EPM) of male and female mice on unsupplemented diets or diets supplemented with one or more prebiotic or maltodextrin as described in Example 2;

FIG. 8a shows depressive like behavior after a forced swim test of male and female mice on unsupplemented diets or diets supplemented with one or more prebiotic or maltodextrin as described in Example 2; and

FIG. 8b shows statistically significant sample results of depressive like behavior measured in FIG. 8a and as described in Example 2.

### DETAILED DESCRIPTION

For purposes of the following description, the use of “or” means “and/or” unless specifically stated otherwise, even though “and/or” may be explicitly used in certain instances.

As used herein, “including,” “containing” and like terms are understood in the context of this application to be synonymous with “comprising” and are therefore open-ended and do not exclude the presence of additional undescribed or unrecited elements, materials, ingredients or method steps.

As used herein, “consisting of” is understood in the context of this application to exclude the presence of any unspecified element, ingredient or method step.

As used herein, “consisting essentially of” is understood in the context of this application to include the specified elements, materials, ingredients or method steps “and those that do not materially affect the basic and novel characteristic(s)” of what is being described.

As used herein, “subject” or “individual” means animals, including mammals, including humans, a canine, a feline, a bovine, an equine, a porcine, a primate, and/or a rodent. Also, as used herein, “administering” an amount (e.g., a dose) of a composition may be done by the subject himself/herself or another subject (e.g., a medical professional, a caretaker, a family member). The composition may be provided to the subject or the administrator for the subject along with instructions for administration of the composition (e.g., written instructions on a label of a container containing the composition).

A composition is described. The composition includes, consists essentially of or consists of at least one prebiotic or at least two prebiotics selected from a source of pectin, a source of beta-glucan, a source of xylooligosaccharide and Ashwagandha that is suitable for administration to a subject (e.g., a human subject). The composition may be in the form of a dietary or nutritional supplement that is added to a diet (supplements a normal human diet). The composition includes at least two prebiotics in an amount to increase production of (A) tryptophan or (B) tryptophan and/or at least one of (i) indole, (ii) an indole derivative(s), (iii) 2,3-pyridinecarboxylic acid (iv) 3-(4-hydroxyphenyl) propionic acid and (v) a short chain fatty acid(s) and/or (C) any other metabolite that benefits brain health, particularly mood (e.g., serotonin, dopamine, gamma aminobutyric acid (GABA)) in a subject. By increasing (A) tryptophan or (B) tryptophan and/or at least one of (i) indole, (ii) an indole derivative(s), (iii) 2,3-pyridinecarboxylic acid (iv) 3-(4-hydroxyphenyl) propionic acid and (v) a short chain fatty acid(s) and/or (C) any other metabolite that benefits brain health, particularly mood (e.g., serotonin, dopamine, GABA), the composition has a beneficial effect on mood, particularly a reduction in stress or anxiety and/or depression. A method for improving mood such as reducing stress oranxiety and/or depression is also described. The method includes administering a daily dosage amount of a composition comprising, consisting essentially of or consisting of at least one prebiotic or at least two prebiotics selected from a source of pectin, a source of beta-glucan, a source of xylooligosaccharide and Ashwagandha (an herb) in an amount to increase production of (A) tryptophan or (B) tryptophan and/or at least one of (i) indole, (ii) an indole derivative(s), (iii) 2,3-pyridinecarboxylic acid (iv) 3-(4-hydroxyphenyl) propionic acid and (v) a short chain fatty acid(s) and/or (C) any other metabolites that benefits brain health (e.g., serotonin, dopamine, GABA). The gut-brain axis of a subject (e.g., a human subject) promotes a bi-directional communication between emotional and cognitive centers and the gastrointestinal system, residence of trillions of microbes. Microbial metabolites generated in human gut are absorbed and play a crucial role in most biological processes including brain function. Serotonin is a key neurotransmitter, which is synthesized in the periphery within the gut neurons and enterochromaffin cells and centrally within the neurons in the brain stem. It influences mood (stress, anxiety, depression). Serotonin is also a precursor for melatonin (a hormone which influences sleep). 90 percent of serotonin is synthesized in the gut. Bacterial products such as short chain fatty acids, especially butyrate can upregulate serotonin production by the enterochromaffin cells.

Tryptophan is a precursor for serotonin synthesis and therefore, may affect serotonin levels. Tryptophan is an amino acid utilized to synthesize proteins; however, intestinal bacteria can directly utilize tryptophan to produce many immunologically important metabolites such as indole, indolic acid derivatives and tryptamines in the gut. Many bacterial species can convert tryptophan into indole and indole derivatives through an enzyme, tryptophanase. The role of indole and its derivatives is an emerging area of research and there is some evidence of their beneficial effects. Therapeutic administration of oral indole propionic acid was protective in a murine model of colitis suggesting an anti-inflammatory role. Indole acetic acid (IAA) was also shown to alleviate high fat diet-induced hepatotoxicity in mice.

By leveraging the effects of changes in microbiome and the production of tryptophan, indole, indole metabolites, 2,3-pyridinecarboxylic acid, 3-(4-hydroxyphenyl) propionic acid and/or small chain fatty acids and/or any other metabolite relevant to brain health with individual prebiotics or combinations of two or more prebiotics of pectin, beta-glucan, a xylooligosaccharide and Ashwagandha, can provide benefits to mammals particularly related to brain health, especially to mood in the sense of a reduction of stress, anxiety and/or depression. Microbial metabolites like short chain fatty acids, tryptophan, indole metabolites have potential to particularly benefit mood through alleviation of stress induced anxiety and/or depression through the gut-brain axis. While the effect of the identified prebiotics (a source of pectin, a source of beta-glucan, a source of xylooligosaccharide and Ashwagandha) administered to a subject singly particularly benefits mood, the effect of combinations of the identified prebiotics (e.g., two or more prebiotics administered to a subject so as to be present in the gut of the subject at the same or similar times) is believed to be different relative to the effect of prebiotics administered singly as the combinations are believed to provide competition for metabolizing machinery of gut microbes. The presence of more than one prebiotic in the gut of a subject simultaneously (at the same or similar times) is believed to diversify microbial composition and metabolites, which may provide similar as well as additional health benefits, particularly regarding mood benefits in terms of reducing stress/anxiety/depression.

Although reference is made herein to “a” source of pectin, “a” source of beta-glucan, “a” source of xylooligosaccharide, “an” Ashwagandha, a combination (i.e., a plurality) of these components can be used (e.g., two or more of sources or forms of a pectin, two or more sources or forms of a beta-glucan, two or more sources or forms of a xylooligosaccharide and two or more sources or forms of Ashwagandha).

Pectin is a soluble fiber found in high levels in apples, plums and the peels of citrus fruits. In vitro assays showed that human gut microbes cultured with purified (approximately 95 percent or greater) apple pectin having a degree of esterification on the order of 68 percent to 78 percent (e.g., commercially available from Herbstreith & Fox GmbH & Co.) increased tryptophan and indole production. A suitable source of pectin may include at least 75 percent to 95 percent or more pectin by weight.

Beta-glucan is also a soluble fiber that is found in whole grains, oats, bran, wheat and barley. One suitable beta-glucan in the described dietary or nutritional supplement composition is a beta-glucan derived from oats that is composed of beta-1, 3 linkages and beta-1, 4 linkages. This type of beta-glucan is also referred to as a mixed-linkage (1→3), (1→4)-beta-D-glucan, A purified source of beta-glucan derived from oats extract containing at least 75 percent by weight beta-glucan may be suitable in the described dietary or nutritional supplement composition, such as 90 percent by weight or more, such as 95 percent by weight or more. In vitro assays showed that human gut microbes cultured with oats beta-glucan obtained from Guangzhou Sinacon Food Ltd. increased tryptophan, indole metabolites (derivatives), 2,3-pyridinecarboxylic acid and 3-(4-hydroxyphenyl) propionic acid production.

Xylooligosaccharides (XOS) are oligomers of sugars, formed by xylose units, which are non-caloric and generally not digestible by humans In vitro assays showed that human gut microbes cultured with a purified source of xylooligosaccharides derived from corn extract having XOS greater than 90 percent by weight (e.g., commercially available from AIDP (PreticX™) increased tryptophan production. The purified source of xylooligosaccharides also has a bifidogenic effect (increase in Bifidobacterium) and a butyrogenic effect (increase in butyrate). A source of xylooligosaccharides that contains at least 75 percent by weight xylooligosacchirdes may be suitable in the described dietary or nutritional supplement composition, such as 90 percent by weight or more, such as 95 percent by weight or more.

Ashwagandha (Withania somnifera) is a plant or herb containing withanolides. In vitro assays demonstrated that an Ashwagandha extract containing greater than 10 percent concentration of withanolides by weight (e.g., Sensoril® from Natreon Inc.) has a bifidogenic effect (increase in Bifidobacterium) and a butyrogenic effect (increase in butyrate).

Based on the in vitro studies of the noted prebiotics, consumption of a combination of two or more prebiotics of a source of pectin, a source of beta-glucan, a source of xylooligosaccharide and Ashwagandha in the form of a dietary supplement will increase either gut microbial tryptophan or increase gut microbial tryptophan and/or one or more of indole, an indole derivative, 2,3-pyridinecarboxylic acid (iv), 3-(4-hydroxyphenyl) propionic acid and (v) a short chain fatty acid and/or any other metabolite relevant to brain health (e.g., dopamine, serotonin, GABA). Although reference is made to “an” indole derivative and “a” short chain fatty acid, a combination (i.e., a plurality) of these components can be present (e.g., two or more indole derivatives, two or more short chain fatty acids). Examples of indole derivatives include but are not limited to indole-3-propionic acid, indole-3-butyric acid, indole-3-acetic acid and indole-3-pyruvic acid. Examples of short chain fatty acids include but are not limited to acetate, propionate and butyrate. The increased tryptophan production or increased tryptophan and/or one or more of indole, an indole derivative(s), 2,3-pyridinecarboxylic acid (iv) 3-(4-hydroxyphenyl) propionic acid and a short chain fatty acid(s) production and/or any other metabolite relevant to brain health will provide benefits to humans particularly related to brain health and more particularly to mood in the sense of a reduction of stress, anxiety and/or depression. Some of the other metabolites produced in the gut such as serotonin, dopamine and GABA also beneficially influence brain function including mood.

A composition as a supplement (a nutritional or dietary supplement) includes an amount of two or more prebiotics selected from a source of pectin, a source of beta-glucan, a source of xylooligosaccharide and Ashwagandha in an amount to increase production of either (A) tryptophan or (B) tryptophan and/or at least one of (i) indole, (ii) an indole derivative(s), (iii) 2,3-pyridinecarboxylic acid (iv) 3-(4-hydroxyphenyl) propionic acid and (v) a short chain fatty acid(s) and/or (C) any other metabolite that benefits brain health, particularly mood (e.g., serotonin, dopamine, GABA). A representative daily dosage for an adult human of a source of pectin (e.g., 95 percent pectin or greater) is 4 to 10 grams, such as 5 to 6 grams. A representative daily dosage of a source of beta-glucan (e.g., an extract containing at least 85 percent beta-glucan) is 2 to 6 grams such as 3 to 4 grams. A representative dosage of a source of xylooligosaccharide extract (e.g., an extract containing at least 90 percent xylooligosaccharide) is 2 to 6 grams, such as 3 to 4 grams. A representative daily dosage of Ashwagandha is 100 to 200 milligrams, such as 100 to 175 milligrams, such as 125 to 150 milligrams. The daily dosage may be consumed in as a single composition at one time (once daily) or as divided composition with portions of the composition consumed throughout the day (e.g., twice daily, three times daily).

Representative compositions as a supplement include a composition including a source of pectin (e.g., apple pectin), a source of beta-glucan (e.g., oats beta-glucan), a source of xylooligosaccharide and/or Ashwagandha; a composition including a source of pectin (e.g., apple pectin), a source of beta-glucan (e.g., oats beta-glucan) and a source of xylooligosaccharide; a composition including a source of pectin (e.g., apple pectin), a source of beta-glucan (e.g., oats beta-glucan) and Ashwagandha; a composition including a source of pectin (e.g., apple pectin) and a source of xylooligosaccharide; a composition including a source of beta-glucan (e.g., oats beta-glucan) and a source of xylooligosaccharide; a composition including a source of pectin (e.g., apple pectin) and Ashwagandha; and a composition including a source of beta-glucan (e.g., oats beta glucan) and Ashwagandha.

A composition as a supplement (a nutritional or dietary supplement) that includes an amount of two or more prebiotics selected from a source of pectin, a source of beta-glucan, a source of xylooligosaccharide and Ashwagandha in an amount to increase production of (A) tryptophan or (B) tryptophan and/or at least one of (i) indole, (ii) an indole derivative(s), (iii) 2,3-pyridinecarboxylic acid (iv) 3-(4-hydroxyphenyl) propionic acid and (v) a short chain fatty acid(s) and/or (C) any other metabolite relevant to brain health (e.g., dopamine, serotonin, GABA) may be produced by combining the two or more prebiotics in dry, e.g., powder, form and mixing. The mixture may then be weighed and packaged in a dosage form that includes, but is not limited to, tablets, controlled release tablets, chewing tablets, enteric coated tablets, gummy compositions, capsules, solutions (e.g., beverages), syrups or powder to be added to a liquid (a liquid miscible powder) depending on the proposed application. Some dosage forms may be formulated together with, for example, diluents, excipients or carriers and disintegrants. The package may include printed instructions that direct an individual on a recommended dosage (e.g., a recommended daily dosage) to supplement a diet (e.g., supplement the food otherwise taken in the form of meals (e.g., breakfast, lunch and/or dinner) or snacks) and a dosage preparation technique if applicable.

A dietary or nutritional supplement composition including one or more prebiotics selected from a source of pectin, a source of beta-glucan, a source of xylooligosaccharide and Ashwagandha in an amount to increase production of (A) tryptophan or (B) tryptophan and/or at least one of (i) indole, (ii) an indole derivative(s), (iii) 2,3-pyridinecarboxylic acid (iv) 3-(4-hydroxyphenyl) propionic acid and (v) a short chain fatty acid(s) and/or (C) any other metabolite relevant to brain health (e.g., dopamine, serotonin, GABA) may also include one or more vitamins (e.g., Vitamin B6, Vitamin B12, folic acid), minerals (e.g., magnesium) or other dietary aids (e.g., botanicals (e.g., bacopa)).

### Example 1

The effects of the addition of prebiotics on human gut microbiota composition and microbial metabolite production was evaluated in an in vitro system. Culturing human gut microbes with apple pectin, oats beta-glucan and xylooligosaccharides (XOS) from the cob of corn and Ashwagandha showed their individual capacity to modulate gut microbiota in a distinct manner thereby stimulating growth of different genera of beneficial bacteria—apple pectin (Bifidobacterium, Bacteroides, Faecalibacterium), oats beta-glucan (Roseburia, Blautia), XOS (Blautia, Bifidobacterium) and Ashwagandha (Bifidobacterium) to name a few.

Prebiotic specific changes in gut microbial composition leads to changes in metabolic capacity, which may increase or decrease metabolite production. Consequently, we observed,

an increase in tryptophan production with addition of pectin, e.g., apple pectin (2 fold), beta-glucan (1.4 fold), XOS (1.5 fold); an increase in some of the indole derivatives with addition of oats beta-glucan; and an increase in short chain fatty acids (SCFA) production with apple pectin, oats beta-glucan, XOS and Ashwagandha.

Combining the beneficial effects of changes in microbiome and production of tryptophan, indole, indole metabolites and SCFA produced by the consumption of these prebiotics suggest synergistic effects in mammals (including rodents and humans) related to brain health, particularly benefiting mood through stress, anxiety and/or depression reduction via the gut-brain axis.

Table 1 shows distinct changes in human gut microbial composition and growth stimulation of most abundant beneficial bacteria with the individual addition of apple pectin, beta-glucan, XOS and Ashwagandha.

TABLE 1

| | No | No | A | Oats b- | | |
|---|---|---|---|---|---|---|
| Taxonomy | addition | addition | Pectin | glucan | XOS | Ashwagandha |
| Roseburia | 0.4% | 0.6% | 0.9% | 13.8% | 1.0% | 0.7% |
| Blautia | 5.6% | 5.5% | 6.0% | 12.0% | 16.2% | 7.7% |
| Bifidobacterium | 16.9% | 20.6% | 14.4% | 9.9% | 33.5% | 30.2% |
| Bacteroides | 11.1% | 11.5% | 21.9% | 8.1% | 3.3% | 6.3% |
| Dorea | 8.2% | 7.6% | 7.2% | 8.0% | 7.3% | 9.0% |
| Sutterella | 8.6% | 8.8% | 4.8% | 6.6% | 4.4% | 6.0% |
| Allisonella | 10.9% | 9.8% | 6.1% | 6.2% | 3.8% | 4.1% |
| Collinsella | 4.5% | 2.4% | 1.5% | 5.6% | 8.4% | 6.9% |
| Megasphaera | 4.6% | 6.0% | 6.6% | 4.7% | 6.3% | 7.8% |
| Unclassified_Lachnospiraceae | 5.6% | 4.9% | 5.0% | 3.8% | 2.7% | 2.7% |
| Faecalibacterium | 2.0% | 2.3% | 9.6% | 2.8% | 2.0% | 4.0% |
| Clostridium_XlVa | 3.8% | 3.5% | 2.0% | 2.7% | 2.2% | 1.2% |
| Oscillibacter | 2.8% | 2.2% | 1.6% | 2.3% | 0.7% | 0.5% |
| Coprococcus | 2.3% | 1.9% | 1.7% | 1.9% | 2.0% | 2.4% |
| Mogibacterium | 1.8% | 2.2% | 1.4% | 1.6% | 0.3% | 0.3% |
| Paraprevotella | 1.7% | 1.7% | 1.4% | 1.5% | 1.0% | 1.5% |
| Phascolarctobacterium | 1.0% | 0.8% | 1.3% | 1.3% | 0.7% | 0.6% |
| Clostridium_XlVb | 2.7% | 2.2% | 2.1% | 1.1% | 0.028% | 0.1% |
| Bilophila | 1.1% | 1.0% | 0.5% | 0.8% | 0.0% | 0.016% |
| Fusicatenibacter | 0.024% | 0.028% | 0.064% | 0.752% | 0.856% | 1.795% |

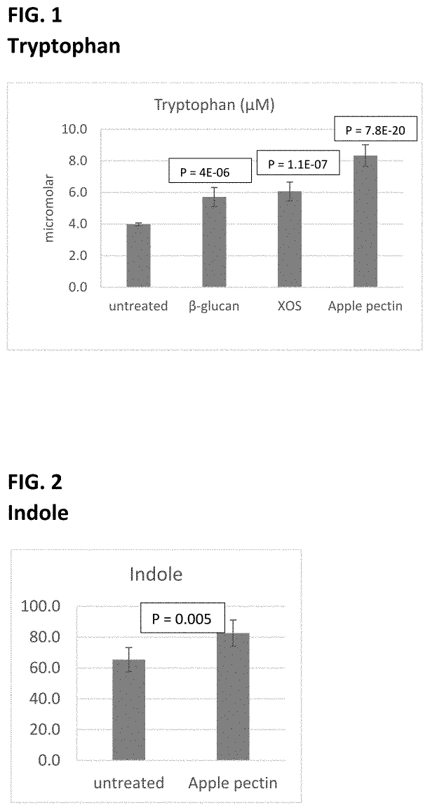

FIG. 1 shows the effect on gut microbial tryptophan production when human gut microbiota was individually cultured with oats beta-glucan, XOS and apple pectin. FIG. 1 shows that an increase in tryptophan production was observed with the addition of oats beta-glucan, XOS and apple pectin.

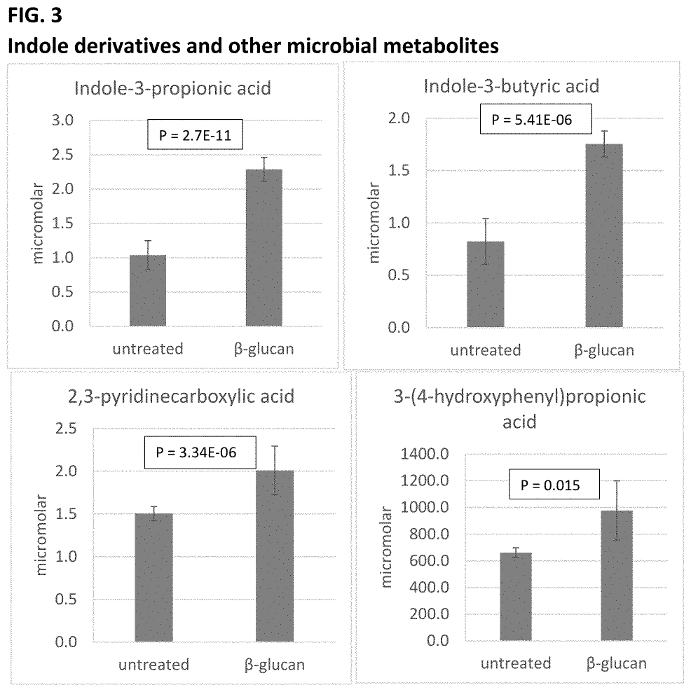

FIG. 2 shows the effect on gut microbial indole production when human gut microbiota was cultured with apple pectin. FIG. 3 shows the effect on the production of gut microbial indole derivatives and other microbial metabolites such as 2,3-pyridinecarboxylic acid and 3-(4-hydroxyphenyl) propionic acid when human gut microbiota was cultured with beta-glucan. FIGS. 2 and 3 show that there was a statistically significant increase in indole production with apple pectin and elevation in the levels of other indole derivatives with beta-glucan addition.

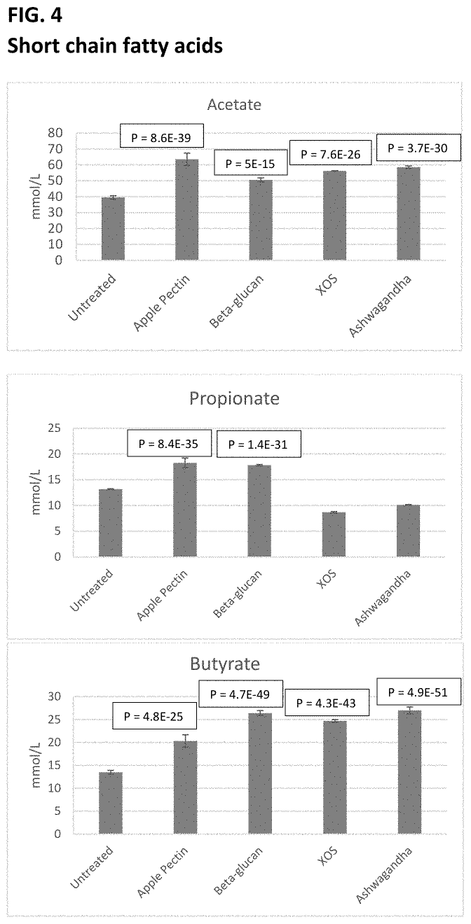

FIG. 4 shows the effect on gut microbial short chain fatty acid production when human gut microbiota was cultured individually with apple pectin, beta-glucan, xylooligosaccharides and Ashwagandha. FIG. 4 shows there was an increase in the production of most SCFA evaluated (acetate, propionate and butyrate) with addition of each probiotic.

### EXAMPLE 2

It is believed that the gut microbiota acts through direct production of neuroendocrine metabolites (hormone-like metabolites such as short chain fatty acids, neurotransmitters, gastrointestinal hormones, precursors to neuroactive compounds such as tryptophan) and/or, indirectly, as the modulator of inflammatory responses, immune responses and hormonal secretion.

Hypothalamic-Pituitary-Axis (HPA): The anatomical structures that mediate the stress response are found in both the central nervous system and peripheral tissues. The principal effectors of the stress response are localized in the paraventricular nucleus (PVN) of the hypothalamus, the anterior lobe of the pituitary gland, and the adrenal gland. This collection of structures is commonly referred to as the hypothalamic-pituitary-adrenal (HPA) axis. HPA is one of the main neuroendocrine systems in mammals. Activation of the HPA axis is a tightly controlled process that involves a wide array of neuronal and endocrine systems. It is subject to feedback inhibition from circulating glucocorticoids. The HPA axis mounts a defensive response when it perceives a threat which ultimately induces the release of behavior-altering hormones such as glucocorticoids, mineral corticoids and catecholamines HPA dysfunction is also associated with depression.

Corticosterone: Glucocorticoids, cortisol in humans and corticosterone in rodents, are endogenous steroid hormones secreted by the adrenal glands under the regulation of the HPA and have pleiotropic functions involved in the stress response, energy metabolism, reproductive function, and inflammatory and immune responses.

Cortisol or corticosterone are important mediators of the stress system. The corticosteroid hormones operate in concert with catecholamines and other transmitters. Insufficient corticosteroid control leads to aggravated stress reactions. Alternatively, if adaptation to stress fails, circulating corticosteroid levels remain elevated for a prolonged period of time. Both, too low and too high cortisol/corticosterone concentrations are detrimental for brain processes.

Stress leads to elevation of corticosterone, which then binds to and activates both mineralocorticoid receptors (MRs) and glucocorticoid receptors (GRs). Since MRs have a higher affinity, these receptors tend to remain occupied even at low, basal levels of corticosterone. In contrast, GRs have a lower affinity for corticosterone; therefore, levels of GR activation tend to correlate with changing levels of free corticosterone. The expression pattern of MRs and GRs differ substantially throughout the brain. MRs are expressed in limbic brain circuits and as well as in the pituitary whereas GRs are more widely expressed throughout the brain. However, GR expression is particularly high in the hypothalamus, hippocampus and the pituitary, suggesting that this receptor plays an important role in regulating the function of the brain cells in these regions.

Stress and Inflammation: A meta-analysis showed statistically significant stress-related increases in circulating interleukins IL-1β, IL-6, IL-10, and tumor necrosis factor (TNFα) but not IL-1ra, IL-2, interferon-c, or C-reactive protein (Marsland et al. Brain, Behavior, and Immunity, 64 2017, 208-219). Stress responses of inflammatory markers have been found to be higher in individuals with different forms of depression. Laboratory studies showed that acute stress was associated with significant increases in IL-1β, and TNFα with IL-b, IL-6, and TNFα demonstrating the most robust increases. Higher cytokine levels were reported to be associated with increases in negative mood and anxiety in some studies.

Gut microbiota also mediates the production of immune mediators such as TNFα, IL-1β and IL-6 that, in turn, reach the brain and stimulate the HPA axis. Moreover, the gut microbiota is able to directly influence the production of glucocorticoid hormones. Research has shown that the interaction between bacterial products and Toll-like receptors (TLR) expressed on the intestinal epithelium is crucial for maintaining homeostatic levels of corticosterone, in fact microbiota—depleted mice have exaggerated and sustained synthesis of corticosterone throughout the day (Mukherji et al., 2013, Cell 153, 812-827).

Behavior assays in rodents: Several behavioral assays have been developed to study stress, anxiety, and depression in rodents. The forced swim test (FST) measures the presence of or reduction in positive coping skills in rats and mice. When the animals are treated with anti-depressant prior to the test, they show reduced immobility and more climbing, suggesting that the animals do not give up, while untreated animals show increased immobility and will float more in the water. Therefore, reduced immobility and more climbing are thought to be an anti-depressive, positive coping phenotype in the FST. Many tests are based on the fact that normal rodents prefer “unexposed” areas. In the elevated plus maze (EPM) test, the frequency and duration that rodents explore in an exposed environment is measured. Anxiety is analyzed by the average time spent in open arms, closed arms, and the center zone of the maze and reduced time spent on open arms indicates that the animal experienced more anxiety.

Experimental Design

This study was designed to evaluate the effect of prebiotic(s) on stress induced anxiety and/or depression. Both behavior and quantitative assessments were done to evaluate physiologically relevant effects. As noted, stress leads to increase in corticosterone and inflammatory cytokines. Resistance to such an increase may lead to positive coping skills and could be seen in behavior tests such as EPM (for anxiety) and FST (for depression).

13 males and 13 female C57BL/6J mice were recruited to each diet group (Table 2). The animals were given immediate ad libitum access to water and standard rodent chow (control diet) and were acclimated to the facility for 7 days. On study day 0, the animals were weighed, blood and fecal samples (1-2 pellets/animal) were collected, and the animals were randomized by weight into their specific diet and treatment groups that included chow only (control group), chow supplemented with maltodextrin or chow supplemented with one or more prebiotics (Table 2). Blood and fecal collections were done at 0, 4 and 8 weeks. On study day 50, animals assigned to the acute stress groups were acutely restrained for 2 hours while unstressed animals were housed singly for 140 minutes in fresh cages. Following the 2-hour stress period, stressed animals were released into their cage (without bedding) and had a 20-minute grooming break. Similarly, on study days 51 and 52, all animals were stressed as above (or not) and then tested on the respective days in the EPM and FST. A retro-orbital blood sample was collected within 15 to 30 minutes after the FST, and fecal pellets were collected at the end of the experiment. Necropsy was performed on study day 56. Blood and tissues were collected for biochemical analysis.

TABLE 2
Diets used in the Example 2

| | | | | | | Mouse equivalent Dose | |
|---|---|---|---|---|---|---|---|
| Group | n | sex | Diet | Diet name | Diet # | (mg/Kg/day) | Treatment |
| 1 | 13/13 | F/M | Prebiotic | P | Diet 1 | 1138.5 | Stress |
| 2 | 13/13 | F/M | Prebiotic | G | Diet 2 | 569.2 | Stress |
| 3 | 13/13 | F/M | Prebiotic | X | Diet 3 | 569.2 | Stress |
| 4 | 13/13 | F/M | Prebiotic | A | Diet 4 | 28.5 | Stress |
| 5 | 13/13 | F/M | Prebiotics | PX | Diet 5 | 1138.5, 569.2 | Stress |
| 6 | 13/13 | F/M | Prebiotics | GX | Diet 6 | 569.2, 569.2 | Stress |
| 7 | 13/13 | F/M | Prebiotics | PA | Diet 7 | 1138.5, 28.5 | Stress |
| 8 | 13/13 | F/M | Prebiotics | GA | Diet 8 | 569.2, 28.5 | Stress |
| 9 | 13/13 | F/M | Prebiotics | PG | Diet 9 | 1138.5, 569.2 | Stress |
| 10 | 13/13 | F/M | Prebiotics | PGX | Diet 10 | 1138.5, 569.2, 569.2 | Stress |
| 11 | 13/13 | F/M | Prebiotics | PGA | Diet 11 | 1138.5, 569.2, 28.5 | Stress |
| 12 | 13/13 | F/M | Non-prebiotic diet | Maltodextrin | Diet 12 | 1707.7 | Stress |
| 13 | 13/13 | F/M | Control diet | Diet | Diet 13 | 0 | Stress |
| 14 | 13/13 | F/M | Control diet | Diet | Diet 13 | 0 | No Stress |

P = Apple pectin,
G = Oats beta glucan,
X = Xylooligosaccharides,
A = Ashwagandha

Results

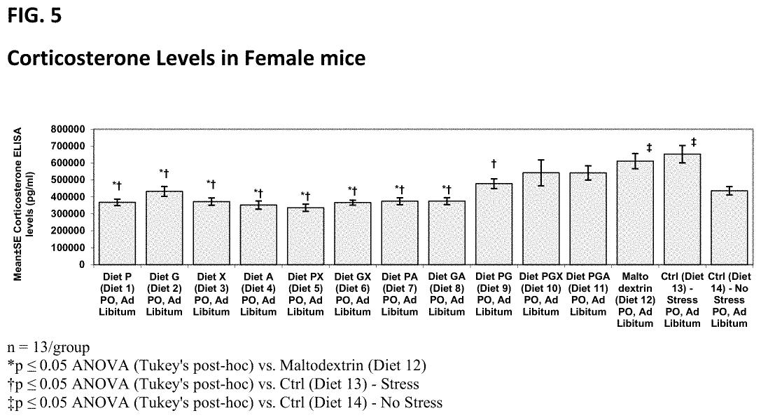

Endocrine response: Alterations in the hypothalamic-pituitary-1adrenal (HPA) axis and stress response has been linked to the development of mood disorders. Serum corticosterone levels were measured after acutely stressed mice underwent FST. After FST, the serum corticosterone levels were increased in stressed female mice and were not reduced by maltodextrin (non-prebiotic) supplementation. However, serum corticosterone levels in female mice were statistically reduced (F(3, 48)>7.1, p<0.0005) in most prebiotic treatment groups (except diets apple pectin/oats beta glucan/xylooligosaccharides (PGX) and apple pectin/oats beta glucan/Ashwagandha (PGA)) as compared to the stressed control and as compared to maltodextrin controls except the diet of apple pectin/oats beta glucan (PG) (FIG. 5). Moreover, the levels were reduced to that of the unstressed mice. This suggests that diets of one of apple pectin (P), oats beta glucan (G), xylooligosaccharides (X), Ashwagandha (A), Apple pectin/xylooligosaccharides (PX), oats beta glucan/xylooligosaccharides (GX), apple pectin/Ashwagandha (PA), oats beta glucan/Ashwagandha (GA) or apple pectin/oats beta glucan (PG) lead to better adaptation to stress response. There was also a greater reduction in oats beta glucan/ xylooligosaccharides (GX) and oats beta glucan/Ashwagandha (GA) as compared to oats beta glucan (G) alone. Male mice did not show the same reduction in corticosterone levels in stressed mice as female mice with prebiotic treatment (data not shown).

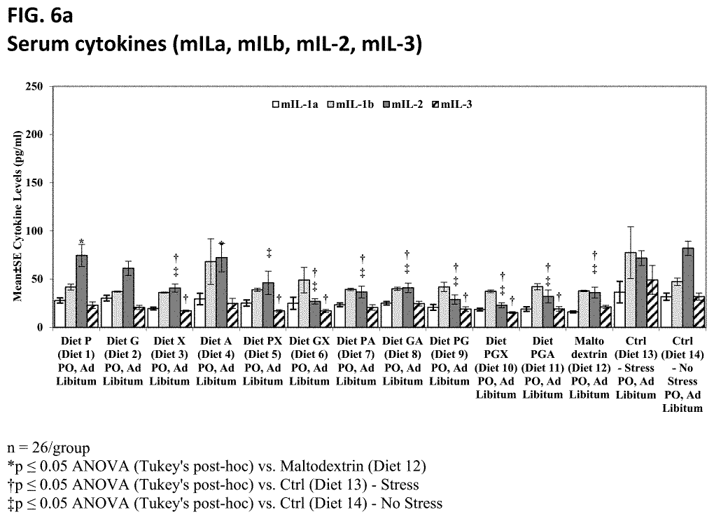

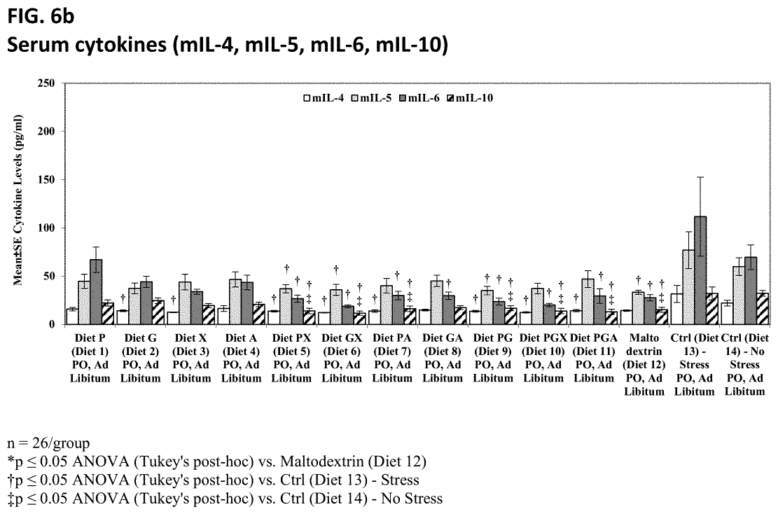

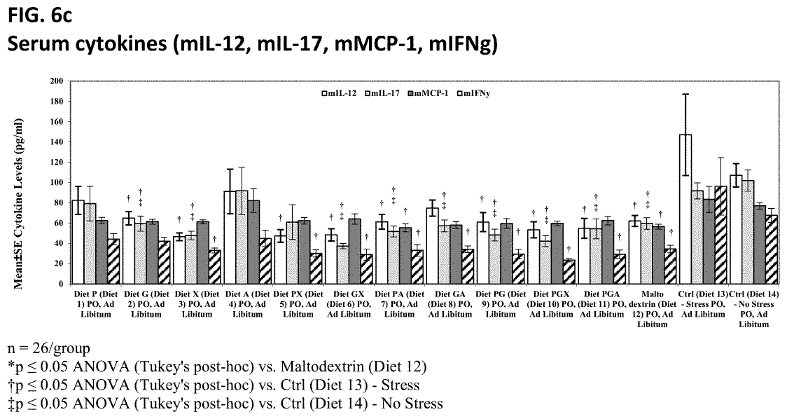

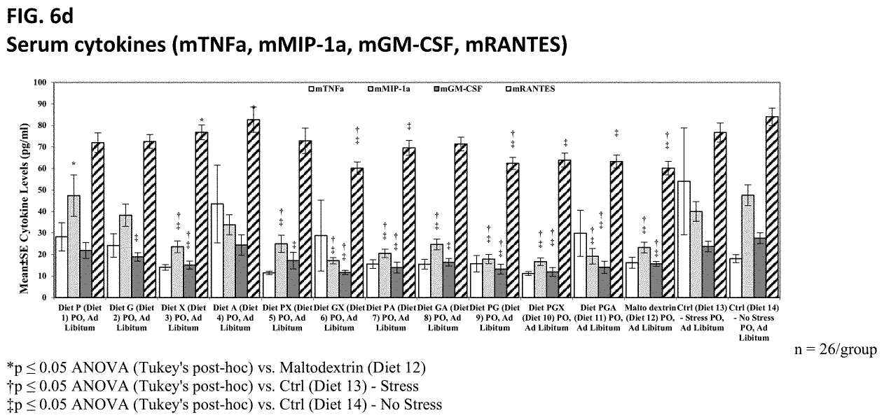

Inflammatory cytokine levels: A panel of 16 serum cytokines was analyzed using blood collected after 8 weeks of dietary supplementation at the termination of the experiment. A combined group of male and female mice on the control diet which were stressed acutely during the experiments had higher inflammatory cytokine levels as compared to unstressed control mice. Addition of prebiotic(s) to the diet showed significantly reduced levels for most cytokines as compared to stressed mice indicating potential for reduced systemic inflammation as shown in Table 3 (top panel). Averages of cytokine levels from 26 mice are shown and the graphs in FIG. 6a, FIG. 6b, FIG. 6c and FIG. 6d. Combinations of two or more prebiotics were more effective in reducing the cytokine levels than individual dietary prebiotic treatment as shown in Table 3 (bottom panel) representing averages for cytokines for individual prebiotics, double combinations and triple combinations, which helps visualize the trend.

Surprisingly, treatment with a maltodextrin (DE-13) supplemented diet that was used as non-prebiotic control also showed reduction in inflammatory cytokines, which could be due to a distinct mechanism. Maltodextrin is a polysaccharide derived from starch consisting of D-glucose units linked primarily by α-1-4 bonds and that has a dextrose equivalent (DE) of less than 20. Commonly, maltodextrin sold in the market has a DE between 3 and 20. The higher the DE value, the shorter the glucose chains. DE>20 is called glucose syrup. DE13-17 contains more low-molecular glucose units and less high-molecular glucose units. These glucose units may be acted upon by a-amylase, converted to maltose and are rapidly absorbed in the small intestines unlike prebiotics, which remain undigested in upper gastrointestinal tract and are fermented in the colon by gut bacteria.

TABLE 3
Average cytokines in combined males and females.

| Diet | Diet | Diet | Diet | Diet | Diet | Diet | Diet | Diet | Diet | |
|---|---|---|---|---|---|---|---|---|---|---|
| PGX | PG | GA | PA | GX | PX | A | X | G | P | |
| 18.48 | 20.76 | 25.06 | 23.39 | 24.94 | 25.20 | 29.38 | 19.47 | 30.29 | 27.90 | IL-1a |
| 37.35 | 41.95 | 39.97 | 39.26 | 48.98 | 38.92 | 68.21 | 36.09 | 37.10 | 41.82 | IL-1b |
| 22.91 | 28.90 | 40.99 | 36.63 | 26.91 | 46.15 | 72.31 | 40.77 | 61.26 | 74.57 | IL-2 |
| 15.34 | 18.92 | 24.59 | 20.61 | 17.04 | 17.03 | 24.83 | 17.11 | 20.68 | 22.93 | IL-3 |
| 12.42 | 13.58 | 14.88 | 13.85 | 12.20 | 13.68 | 16.46 | 12.54 | 14.08 | 15.82 | IL-4 |
| 37.17 | 35.11 | 45.08 | 40.06 | 35.77 | 36.89 | 46.64 | 43.91 | 37.37 | 44.70 | IL-5 |
| 20.06 | 23.65 | 29.66 | 29.90 | 18.73 | 26.67 | 43.50 | 33.86 | 44.09 | 66.99 | IL-6 |
| 13.84 | 16.76 | 17.41 | 16.15 | 11.59 | 13.92 | 20.77 | 19.50 | 24.52 | 22.20 | IL-10 |
| 53.34 | 60.95 | 74.73 | 61.21 | 48.40 | 47.32 | 91.13 | 46.51 | 64.78 | 82.41 | IL-12 |
| 42.21 | 48.18 | 57.25 | 51.69 | 37.31 | 60.89 | 91.84 | 47.72 | 59.44 | 79.11 | IL-17 |
| 59.67 | 59.51 | 58.01 | 55.41 | 64.00 | 62.28 | 82.20 | 61.26 | 61.36 | 62.48 | MCP-1 |
| 23.37 | 29.37 | 34.16 | 33.15 | 28.85 | 30.05 | 44.80 | 33.03 | 42.10 | 44.18 | IFNy |
| 11.14 | 15.68 | 15.47 | 15.55 | 28.78 | 11.46 | 43.46 | 14.01 | 24.20 | 28.18 | TNFa |
| 16.63 | 17.86 | 24.68 | 20.50 | 17.10 | 24.95 | 33.81 | 23.57 | 38.25 | 47.36 | MIP-1a |
| 11.86 | 13.16 | 16.38 | 13.91 | 11.64 | 17.16 | 24.41 | 15.06 | 18.90 | 21.86 | GM-CSF |
| 63.88 | 62.39 | 71.41 | 69.54 | 60.17 | 72.87 | 82.66 | 76.79 | 72.53 | 71.96 | RANTES |

| 3 | 2 | | Ctrl | Ctrl- | |
|---|---|---|---|---|---|
| prebiotics | prebiotics | Individual | Stress | No Stress | |
| 18.71 | 23.87 | 26.76 | 36.52 | 31.78 | IL-1a |
| 39.78 | 41.82 | 45.80 | 77.49 | 47.35 | IL-1b |
| 27.48 | 35.91 | 62.23 | 71.84 | 82.04 | IL-2 |
| 17.26 | 19.64 | 21.39 | 49.20 | 31.87 | IL-3 |
| 13.32 | 13.64 | 14.72 | 31.61 | 22.09 | IL-4 |
| 42.04 | 38.58 | 43.16 | 77.01 | 59.87 | IL-5 |
| 24.73 | 25.72 | 47.11 | 111.66 | 69.60 | IL-6 |
| 13.45 | 15.17 | 21.75 | 32.12 | 32.14 | IL-10 |
| 54.06 | 58.52 | 71.20 | 147.00 | 107.11 | IL-12 |
| 48.26 | 51.06 | 69.53 | 91.73 | 101.85 | IL-17 |
| 61.10 | 59.84 | 66.82 | 83.35 | 76.98 | MCP-1 |
| 26.25 | 31.12 | 41.03 | 96.22 | 67.57 | IFNy |
| 20.50 | 17.39 | 27.46 | 54.01 | 18.07 | TNFa |
| 17.88 | 21.02 | 35.75 | 40.00 | 47.55 | MIP-1a |
| 12.94 | 14.45 | 20.06 | 23.71 | 27.55 | GM-CSF |
| 63.54 | 67.27 | 75.98 | 76.70 | 84.02 | RANTES |

| Ctrl- | Ctrl | | Diet |
|---|---|---|---|
| No Stress | Stress | Maltodextrin | PGA |
| 31.78 | 36.52 | 16.00 | 18.95 |
| 47.35 | 77.49 | 37.76 | 42.22 |
| 82.04 | 71.84 | 36.02 | 32.05 |
| 31.87 | 49.20 | 21.27 | 19.18 |
| 22.09 | 31.61 | 14.28 | 14.23 |
| 59.87 | 77.01 | 33.23 | 46.92 |
| 69.60 | 111.66 | 27.60 | 29.41 |
| 32.14 | 32.12 | 15.21 | 13.05 |
| 107.11 | 147.00 | 62.07 | 54.78 |
| 101.85 | 91.73 | 59.60 | 54.31 |
| 76.98 | 83.35 | 56.39 | 62.53 |
| 67.57 | 96.22 | 34.41 | 29.13 |
| 18.07 | 54.01 | 16.15 | 29.86 |
| 47.55 | 40.00 | 23.25 | 19.13 |
| 27.55 | 23.71 | 15.65 | 14.01 |
| 84.02 | 76.70 | 60.13 | 63.21 |

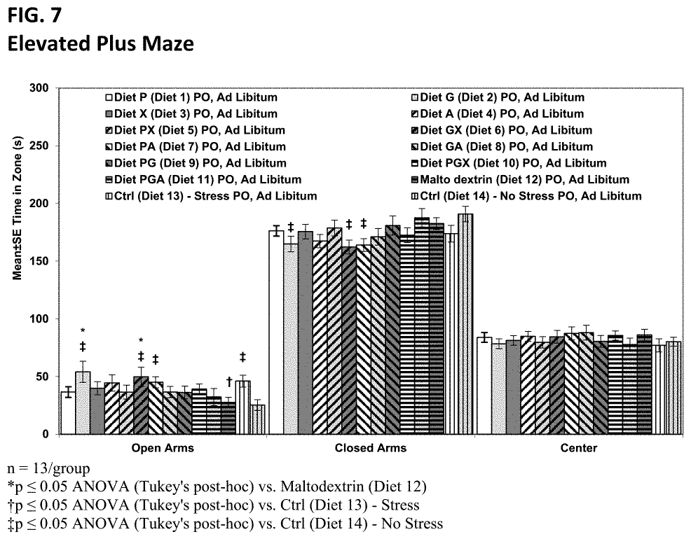

Anxiety like behavior. In the EPM, female mice on diets supplemented with oats beta glucan (G), oats beta glucan/xylooligosaccharides (GX) and apple pectin/Ashwagandha (PA) demonstrated significantly increased open arm times (F(3, 48)>4.5, p<0.0070) and significantly reduced closed-arm times as compared to the unstressed control group with open arm times for mice on diets oats beta glucan (G) or glucan/xylooligosaccharides (GX) also being significant versus the maltodextrin (non-prebiotic) control group of stressed mice (FIG. 7). In the EPM, time spent on open and closed arms for male mice on test diets did not differ statistically from the diet control groups (data not shown).

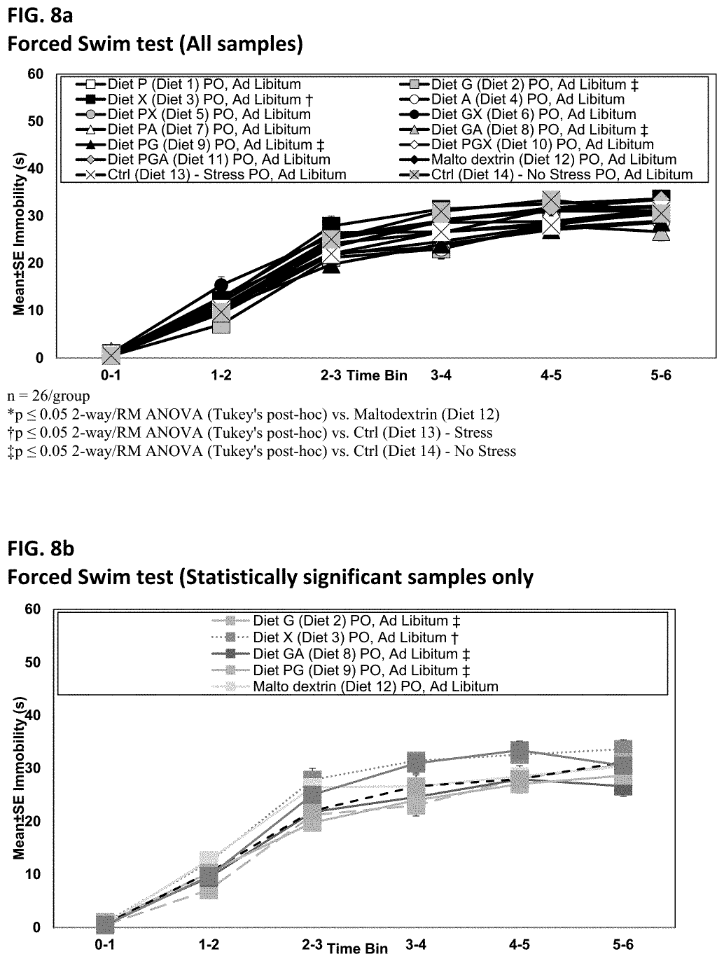

Depressive like behavior: In the FST, immobility over time did not differ statistically across combined female/male control groups. After acute stress, immobility over time was significantly reduced in combined female/male groups treated with diets supplemented with oats beta glucan (G), oats beta glucan/Ashwagandha (GA), or apple pectin/oats beta glucan (PG) as compared to the unstressed control group (Table 4, FIG. 8a, FIG. 8b) and was significantly increased in mice on diet X as compared to the stressed control group (FIG. 8a). Table 4 shows averages of immobility over time within the groups. Comparison of individual versus combined treatments showed that mice on diets including oats beta glucan (G) or apple pectin (PG) had significantly reduced immobility over time as compared to mice on diets including xylooligosaccharides (X), oats beta glucan/xylooligosaccharides (GX), apple pectin/Ashwagandha (PA), apple pectin/oats beta glucan/xylooligosaccharides (PGX) or apple pectin/oats beta glucan/Ashwagandha (PGA). Immobility was also significantly reduced in mice on diets including Ashwagandha (A) or oats beta glucan/Ashwagandha (GA) as compared to mice on diet including apple pectin/oats beta glucan/Ashwagandha (PGA). Although data was not statistically significant as compared to stressed maltodextrin controls, the trend towards reduced immobility (Table 4) was seen indicating potential for positive coping skills.

TABLE 4
Forced Swim Test (averages)

| | | | | Malto- | Ctrl | Ctrl |
|---|---|---|---|---|---|---|
| | Diet G | Diet GA | Diet PG | dextrin | (Diet 13) - | (Diet 14) - |
| Minutes | (Diet 2) ‡ | (Diet 8) ‡ | (Diet 9) ‡ | (Diet 12) | Stress | No Stress |
| 0-1 | 0.5 | 0.9 | 0.9 | 0.6 | 0.7 | 0.5 |
| 1-2 | 7.1 | 9.4 | 10.3 | 12.7 | 10.4 | 9.7 |
| 2-3 | 21.2 | 21.8 | 19.8 | 26.5 | 22.0 | 25.1 |
| 3-4 | 23.0 | 24.6 | 24.0 | 26.6 | 26.6 | 31.0 |
| 4-5 | 28.1 | 28.0 | 27.1 | 28.5 | 28.0 | 33.4 |
| 5-6 | 30.7 | 26.6 | 28.7 | 30.4 | 31.2 | 30.5 |

The experiment presented in Example 2 shows that [1] all diets supplemented with the noted prebiotics showed statistically significant reduction in corticosterone (the stress hormone) in the female mice except triple combinations of PGA and PGX where the reduction was not statistically significant. [2] All prebiotic supplemented diets individually or in combinations reduced stress induced inflammation in both male and female mice. The results also indicate that there was more reduction in diets supplemented with prebiotic combinations than individual prebiotics. [3] The results of behavior outcomes suggest that prebiotic diets supplemented with oats beta glucan (G) or oats beta glucan/xylooligosaccharides (GX) increased time spent on open arms in the EPM when compared to stressed non-prebiotic maltodextrin controls indicating an increase in exploratory behavior and a reduced anxiety phenotype. This is consistent with reduction in corticosterone levels. [4] The results of behavior outcomes also suggest that reduction in immobility in the FST was observed (diets oats beta glucan (G), oats beta glucan/Ashwagandha (GA), and apple pectin/oats beta glucan (PG) when compared to unrestrained control animals. Although the data was not statistically significant as compared to stressed maltodextrin controls, the trends in reduced immobility were seen indicating potential for positive coping skills. [5] Finally, the results indicate that the effect of prebiotic supplementation was more pronounced in females than males.

The mouse equivalent dose presented in Example 2 for each prebiotic provided to the animals individually or in combination with one or more other prebiotic was calculated based on a target human dose of at least: apple pectin (P) 6 grams/day of an apple pectin source of 90 percent apple pectin by weight; oats beta glucan (G) 3 grams/day of an oats beta glucan source of 85 percent by weight oats beta glucan; xylooligosaccharides (X) 3 grams/day of a xylooligosaccharides source of 90 percent by weight xylooligosaccharides; and Ashwagandha (A) 150 milligrams/day.

### Aspects

Aspect 1. A dietary supplement composition comprising, consisting essentially of or consisting of at least one prebiotic or at least two prebiotics selected from a source of pectin, a source of beta-glucan, a source of xylooligosaccharide and Ashwagandha in an amount to increase production of (A) tryptophan or (B) tryptophan and/or at least one of (i) indole, (ii) an indole derivative, (iii) 2,3-pyridinecarboxylic acid (iv) 3-(4-hydroxyphenyl) propionic acid and (v) a short chain fatty acid. and/or (C) any other metabolite relevant to (e.g., benefitting) brain health (e.g., dopamine, serotonin, GABA) such as improving mood by reducing stress/anxiety/ depression in a subject.

Aspect 2. The dietary supplement composition of Aspect 1, wherein the source of pectin comprises apple pectin.

Aspect 3. The dietary supplement composition of Aspect 1 or Aspect 2, wherein the source of beta-glucan comprises beta-glucan derived from oats.

Aspect 4. The dietary supplement composition of any of Aspects 1-3, wherein when the dietary supplement comprises at least two prebiotics, the at least two prebiotics comprise, consist essentially of or consist of the source of pectin, the source of beta-glucan and the source of xylooligosaccharide.

Aspect 5. The dietary supplement composition of any of Aspects 1-3, wherein when the dietary supplement comprises at least two prebiotics, the at least two prebiotics comprise, consist essentially of or consist of the source of pectin, the source of beta-glucan and the Ashwagandha.

Aspect 6. The dietary supplement composition of any of Aspects 1-3, wherein when the dietary supplement comprises at least two prebiotics, the at least two prebiotics comprise, consist essentially of or consist of the source of pectin and the source of beta-glucan.

Aspect 7. The dietary supplement composition of any of Aspects 1-3, wherein when the dietary supplement comprises at least two prebiotics, the at least two prebiotics comprise pectin and the source of xylooligosaccharide.

Aspect 8. The dietary supplement composition of any of Aspects 1-3, wherein when the dietary supplement comprises at least two prebiotics, the at least two prebiotics comprise, consist essentially of or consist of the source of beta-glucan and the source of xylooligosaccharide.

Aspect 9. The dietary supplement composition of any of Aspects 1-3, wherein when the dietary supplement comprises at least two prebiotics, the at least two prebiotics comprise, consist essentially of or consist of the source of pectin and the Ashwagandha.

Aspect 10. The dietary supplement composition of any of Aspects 1-3, wherein when the dietary supplement comprises at least two prebiotics, the at least two prebiotics comprise, consist essentially of or consist of the source of beta-glucan and the Ashwagandha.

Aspect 11. The dietary supplement composition of any of Aspects 1-10, wherein the amount of each prebiotic, if present, in a daily amount, is at least 6 grams of the source of pectin, such as a source of pectin comprising at least 75 percent or at least 95 percent pectin by weight, at least 3 grams of the source of beta-glucan, such as a source of beta-glucan comprising at least 75 percent or at least 85 percent beta-glucan by weight, at least 3 grams of the source of xylooligosaccharide such as a source of xylooligosaccharide comprising at least 75 percent or at least 90 percent xylooligosaccharide by weight and at least 150 milligrams of the Ashwagandha.

Aspect 12. A method for improving mood (reducing stress/anxiety/depression) comprising: administering to a subject a daily dose amount of a composition, e.g., a dietary supplement composition, comprising, consisting essentially of or consisting of at least one prebiotic selected from a source of pectin, source of beta-glucan, a source of xylooligosaccharide and Ashwagandha in an amount to increase production of (A) tryptophan or (B) tryptophan and/or at least one of (i) indole, (ii) an indole derivative, (iii) 2,3-pyridinecarboxylic acid (iv) 3-(4-hydroxyphenyl) propionic acid and (v) a short chain fatty acid and/or (C) any other metabolite (e.g., dopamine, serotonin, GABA) relevant to (e.g., benefitting) brain health such as improving mood in a subject.

Aspect 13. The method of Aspect 12, wherein the source of pectin comprises apple pectin.

Aspect 14. The method of Aspect 12 or Aspect 13, wherein the source of beta-glucan comprises beta-glucan derived from oats.

Aspect 15. The method of any of Aspects 12-14, wherein the at least one prebiotic comprises, consists essentially of or consists of at least two prebiotics.

Aspect 16. The method of any of Aspects 15, wherein the at least two prebiotics comprise, consist essentially of or consist of the source of pectin, the source of beta-glucan and the Ashwagandha or the source of pectin, the source of beta glucan and the source of xylooligosaccharides.

Aspect 17. The method of any of Aspects 15, wherein the at least two prebiotics comprise, consist essentially of or consist of the source of pectin and the source of beta-glucan.

Aspect 18. The method of any of Aspects 15, wherein the at least two prebiotics comprise, consist essentially of or consist of the source of pectin and the source of xylooligosaccharide.

Aspect 19. The method of any of Aspects 15, wherein the at least two prebiotics comprise, consist essentially of or consist of the source of beta-glucan and the source of xylooligosaccharide.

Aspect 20. The method of any of Aspects 15, wherein the at least two prebiotics comprise, consist essentially of or consist of the source of pectin and the Ashwagandha.

Aspect 21. The method of any of Aspects 15, wherein the at least two prebiotics comprise, consist essentially of or consist of the source of beta-glucan and the Ashwagandha.

Aspect 22. The method of any of Aspects 12-21, wherein the daily dosage amount of each prebiotic, if present, is at least 6 grams of the source of pectin, such as a source of pectincomprising at least 75 percent or at least 95 percent pectin by weight, at least 3 grams of the source of beta-glucan, such as a source of beta-glucan comprising at least 75 percent or at least 85 percent beta-glucan by weight, at least 3 grams of the source of xylooligosaccharide such as a source of xylooligosaccharide comprising at least 75 percent or at least 90 percent xylooligosaccharide by weight and at least 150 milligrams of the Ashwagandha.

Whereas specific aspects of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of the invention which is to be given the full breadth of the claims and aspects appended and any and all equivalents thereof.

What is claimed is:

1. A dietary supplement composition comprising at least two prebiotics selected from a source of pectin, a source of beta-glucan, a source of xylooligosaccharide and Ashwagandha in an amount to increase production of (A) tryptophan or (B) tryptophan and/or at least one of (i) indole, (ii) an indole derivative, (iii) 2,3-pyridinecarboxylic acid (iv) 3-(4-hydroxyphenyl) propionic acid and (v) a short chain fatty acid and/or (C) any other metabolite to brain health in a subject.

2. The dietary supplement composition of claim 1, wherein the source of pectin comprises apple pectin.

3. The dietary supplement composition of claim 1, wherein the source of beta-glucan comprises beta-glucan derived from oats.

4. The dietary supplement composition of claim 1, wherein the at least two prebiotics comprise the source of pectin, the source of beta-glucan and the source of xylooligosaccharide.

5. The dietary supplement composition of claim 1, wherein the at least two prebiotics comprise the source of pectin, the source of beta-glucan and the Ashwagandha.

6. The dietary supplement composition of claim 1, wherein the at least two prebiotics comprise the source of pectin and the source of beta-glucan.

7. The dietary supplement composition of claim 1, wherein the at least two prebiotics comprise the source of pectin and the source of xylooligosaccharide.

8. The dietary supplement composition of claim 1, wherein the at least two prebiotics comprise the source of beta-glucan and the source of xylooligosaccharide.

9. The dietary supplement composition of claim 1, wherein the at least two prebiotics comprise the source of pectin and the Ashwagandha.

10. The dietary supplement composition of claim 1, wherein the at least two prebiotics comprise the source of beta-glucan and the Ashwagandha.

11. The dietary supplement composition of claim 1, wherein the amount of each prebiotic, if present, in a daily amount, is at least 6 grams of the source of pectin, at least 3 grams of the source of beta-glucan, at least 3 grams of the source of xylooligosaccharide and at least 150 milligrams of the Ashwagandha.

12. A method for improving mood comprising:
administering to a subject a daily dose amount of a composition comprising at least one prebiotic selected from a source of pectin, a source of beta-glucan, a source of xylooligosaccharide and Ashwagandha in an amount to increase production of (A) tryptophan or (B) tryptophan and/or at least one of (i) indole, (ii) an indole derivative, (iii) 2,3-pyridinecarboxylic acid (iv) 3-(4-hydroxyphenyl) propionic acid and (v) a short chain fatty and/or (C) any other metabolite relevant to brain health.

13. The method of claim 12, wherein the source of pectin comprises apple pectin.

14. The method of claim 12, wherein the source of beta-glucan comprises beta-glucan derived from oats.

15. The method of claim 12, wherein the wherein the at least one prebiotic includes at least two prebiotics.

16. The method of claim 15, wherein the at least two prebiotics comprise the source of pectin, the source of beta-glucan and the Ashwagandha or the source of pectin, the source of beta glucan and the source of xylooligosaccharides.

17. The method of claim 15, wherein the at least two prebiotics comprise the source of pectin and the source of beta-glucan.

18. The method of claim15, wherein the at least two prebiotics comprise the source of pectin and the source of xylooligosaccharide.

19. The method of claim 15, wherein the at least two prebiotics comprise the source of beta-glucan and the source of xylooligosaccharide.

20. The method of claim 15, wherein the at least two prebiotics comprise the source of pectin and the Ashwagandha.

21. The method of claim 15, wherein the at least two prebiotics comprise the source of beta-glucan and the Ashwagandha.

22. The method of claim 12, wherein the daily dosage amount of each prebiotic, if present, is at least 6 grams of the source of pectin, at least 3 grams of the source of beta-glucan, at least 3 grams of the source of xylooligosaccharide and at least 150 milligrams of the Ashwagandha.
